(19) United States
(12) Patent Application Publication
(10) Pub. No.: US 2014/0121497 A1
(43) Pub. Date: May 1, 2014

(54) RADIOFREQUENCY COMPATIBLE AND X-RAY TRANSLUCENT CARBON FIBER AND HYBRID CARBON FIBER STRUCTURES

(71) Applicant: Qfix Systems, LLC, Avondale, PA (US)

(72) Inventors: Daniel D. Coppens, Avondale, PA (US); Nicholas Collura, Philadelphia, PA (US)

(73) Assignee: Qfix Systems, LLC, Avondale, PA (US)

(21) Appl. No.: 14/150,357

(22) Filed: Jan. 8, 2014

Related U.S. Application Data
(63) Continuation-in-part of application No. 13/630,623, filed on Sep. 28, 2012, now abandoned.
(60) Provisional application No. 61/540,488, filed on Sep. 28, 2011.

(51) Int. Cl. A61B 5/055

(52) U.S. Cl. CPC A61B 5/0555
USPC 600/415; 156/60

(57) ABSTRACT

The present disclosure provides a structure constructed of carbon fiber that is compatible with Magnetic Resonance imaging and other radiofrequency technologies. The structure includes carbon fiber elements as well as insulating elements that are substantially x-ray translucent (radiolucent). These elements are arranged in such a way that the structure can be used in modalities such as Magnetic Resonance imaging where carbon fibers typically cannot be used due to image distortion and localized heating. At the same time, the structures are designed to maintain radiolucency that is significantly homogeneous.

### CROSS-REFERENCE TO RELATED APPLICATIONS

This application is a Continuation-in-Part of U.S. patent Ser. No. 13/630,623, filed Sep. 28, 2012 and claims priority to U.S. Provisional Patent Ser. No. 61/540,488 filed Sep. 28, 2011, the entire contents of each are incorporated herein by reference.

### FIELD OF THE TECHNOLOGY

The present disclosure relates to devices designed for Magnetic Resonance (MR) and other radiofrequency (RF) based environments. Specifically, the present disclosure relates to devices comprising carbon fiber that do not cause interference when used in these environments.

### BACKGROUND

Modern Radiation Therapy requires patient positioning devices that are rigid in order to accurately and repeatably position the patient. In addition, the devices must be compatible with the high-energy radiation used during treatment. The unique properties of carbon fiber, high stiffness and radiolucency, have made it an ideal material for patient positioning devices. As state-of-the-art diagnostic imaging technologies are developed and tailored for use in cancer diagnosis and treatment support, the radiolucent properties of carbon fiber have continued to make it the material of choice for modalities such as computed tomography (CT), positron emission tomography (PET), and single-photon emission computed tomography (SPECT), as well as multi-modality imaging techniques such as PET/CT and SPECT/CT in addition to other technologies that are x-ray based.

Generally, treatment of a tumor by radiation therapy is preceded by a diagnostic imaging procedure called simulation. During simulation, the patient is positioned in the manner anticipated for treatment. This includes the physical orientation of the patient using the positioning and immobilization devices that will be used in treatment. This way the computer data set of the patient (DICOM) contains an accurate representation of the location of the tumor. That data set is then imported into treatment planning software (TPS) so that the treatment can be modeled and planned. It is critical that the patient be simulated in the same position on the same devices as will be used in treatment to ensure accurate tumor location identification for treatment.

Magnetic Resonance (MR) imaging provides significant advantages over x-ray based diagnostic imaging techniques in visualizing and differentiating soft tissues such as tumors. There has long been a strong desire to extend the simulation technology to the use of MR imaging. However, until recently, the spatial accuracy of MR machines was not accurate enough for precise tumor location. And precise tumor location is necessary for accurately aiming the treatment beam. In the past, in order to use MR data, the MR data was overlaid or “fused” with CT data to achieve the required accuracy. However, recent advances in spatial accuracy of MR data allow the use of MR information directly for radiation therapy simulation.

MR imaging uses large magnets to create a homogeneous magnetic field. Gradient coils alter the magnetic field in a uniform manner in time or space, creating magnetic field gradients. MR imaging also employs radiofrequency (RF) coils for applying an RF field to a subject to be imaged, causing the resonant nuclei within the subject to resonate and create an MR response signal. An image is then constructed based on this response signal.

Interference with the RF field reduces the quality of the created image. Susceptibility is used to describe the degree of magnetization a material exhibits per applied magnetic field. If a material with susceptibility much different than the subject being imaged is within the magnetic field the homogeneity of the magnetic field will be disturbed near the material. This creates a distortion in the MR image near this material.

Electrically conducting materials, such as metals, disturb and distort the radiofrequency electromagnetic fields necessary for resonance imaging. The eddy currents in these materials, usually metallic conductors of electricity create their own magnetic field that interferes with the fields used for MR imaging. Carbon fiber, which is conductive along its length, also causes this interference.

Other tumor localization techniques also use radiofrequency (RF) technology, such as those techniques developed by Calypso (Seattle, Wash.). For Calypso RF localization to work properly, the accessories cannot interfere with the RF signal generated and reflected by the RF antenna and Beacons respectively. Small conductors do not pose a problem. However, large conductors, such as the metal plates on the end of the Varian Exact™ couch top or sheets of carbon fiber fabric commonly used for patient tables do create signal interference due to eddy current generation.

The electrically conductive nature of carbon fibers is problematic for use in MR imaging machines and other RF devices. Although carbon fiber is not ferro-magnetic, the electrical conductivity can lead to problems such as image distortion and resistance heating of the carbon fiber. The interaction of the carbon fiber with the MR magnetic field causes electrical current to flow through the carbon fibers. This electrical flow can lead to localized magnetic fields as well as localized heating of the material, causing safety concerns. In order to design products that can work in an MRI environment, substitute materials are often used, such as fiberglass and aramid fibers (Kevlar). Although these materials are not conductive, they lack the stiffness of carbon fiber, reducing their applicability to accurate patient positioning during treatment. In the case of fiberglass, the material is not sufficiently radiolucent to be used in significant quantities for structural purposes in an x-ray environment.

The stiffness of commercially available carbon fiber can vary from a modulus of 30 MSI to 120 MSI and greater. As the stiffness increases, the electrical conductivity increases as well. While it can be desirable to make use of these higher stiffness carbon fibers it increases the challenge of incorporating them in MRI compatible structures. This present disclosure makes their use possible.

### SUMMARY

In one embodiment, the present disclosure relates to a structure comprising at least two electrically conductive lamina having carbon fibers embedded in a non-conductive matrix, wherein each conductive lamina has an axis perpendicular to the plane of the lamina (e.g., a vertical axis), and at least one insulating lamina having an axis perpendicular to the plane of the lamina, wherein the conductive lamina are separated by the insulating lamina along the axis perpendicular to the plane of the lamina, wherein the structure is x-ray translucent and does not significantly affect magnetic resonance imaging, x-ray based imaging or other radiofrequency dependent applications. The structures of the present disclosure can be x-ray translucent along the axis perpendicular to the plane of the lamina (e.g., a vertical axis). The structures of the present disclosure can also minimize signal to noise ratio.

In another embodiment, the present disclosure relates to a structure comprising at least two electrically conductive lamina having carbon fiber elements embedded in a non-conductive matrix and insulating elements, wherein each conductive lamina has an axis perpendicular to the plane of the lamina and two in-plane axes, one at zero degrees and one at ninety degrees, wherein the carbon fiber elements are separated by the insulating elements along at least one of the in-plane axes, and at least one insulating lamina having an axis perpendicular to the plane of the lamina, wherein the conductive lamina are separated by the insulating lamina along the axis perpendicular to the plane of the lamina, wherein the structure is x-ray translucent and does not significantly affect magnetic resonance imaging or x-ray based imaging.

In a further embodiment, the present disclosure relates to a structure comprising at least two electrically conductive layers wherein each layer has a plurality of conductive lamina, wherein each conductive lamina has carbon fibers embedded in a non-conductive matrix and an axis perpendicular to the plane of the lamina, and wherein the carbon fibers in any one layer are oriented in substantially the same direction, and at least one insulating lamina having an axis perpendicular to the plane of the lamina, wherein the layers of conductive lamina are separated by the insulating lamina along the axis perpendicular to the plane of the lamina, wherein the structure is x-ray translucent and does not significantly affect magnetic resonance imaging, x-ray based imaging or other radiofrequency dependent applications.

In another embodiment, the present disclosure relates to a method of preparing a patient positioning device, the method comprising placing on a core at least two electrically conductive lamina having carbon fibers embedded in a non-conductive matrix, wherein each conductive lamina has an axis perpendicular to the plane of the lamina, and placing on the core at least one insulating lamina having an axis perpendicular to the plane of the lamina, wherein the conductive lamina are separated by the insulating lamina along the axis perpendicular to the plane of the lamina, wherein the structure is X-ray translucent, wherein the device does not interfere with magnetic resonance and radiofrequency based diagnostics.

In another embodiment, the present disclosure relates to a method of preparing a patient positioning device, the method comprising placing on a core at least two electrically conductive lamina having carbon fiber elements embedded in a non-conductive matrix and insulating elements, wherein each conductive lamina has an axis perpendicular to the plane of the lamina and a zero degree in plane axis and a ninety degree in plane axis, wherein the carbon fiber elements are separated by the insulating elements along at least one of the zero degree axis and the ninety degree axis, and placing on the core at least one insulating lamina an axis perpendicular to the plane of the lamina, wherein the conductive lamina are separated by the insulating lamina along the axis perpendicular to the plane of the lamina, wherein the structure is X-ray translucent, wherein the device does not interfere with magnetic resonance and radiofrequency based diagnostics. The embodiments of the present disclosure can be used for minimizing signal to noise ratio while not significantly affecting magnetic resonance imaging, x-ray based imaging or other radiofrequency dependent applications.

In some embodiments, the device reduces or eliminates image distortion, local heating or combinations thereof.

The non-conductive matrix can include epoxy, polyester, vinylester, or ceramic. The insulating lamina can include aramid, ultra-high-molecular-weight polyethylene or fiberglass. The x-ray based imaging comprises RF Localization, radiation therapy treatment or diagnostic imaging.

The structures can include insulating elements in each conductive lamina that are off-set from each other in at least one of the zero degree axis and the ninety degree axis such that there are an equal number of insulating elements through an axis perpendicular to the plane of the lamina. This arrangement provides an increase in x-ray translucency homogeneity.

The present disclosure also relates to a patient positioning device comprising any of the structures disclosed herein. In one embodiment, the device can include a core, a top face and a bottom face. At least one of the top or bottom faces, or both, include any of the structures disclosed herein. The core can be a closed-cell foam, open-cell foam, honeycomb, wood or a combination thereof.

### BRIEF DESCRIPTION OF THE DRAWINGS

FIG. 1 illustrates elements composed of conductive fibers in the 0 degree direction.

FIG. 2 illustrates typical geometries of the elements in FIG. 1.

FIG. 3 illustrates insulating the elements.

FIG. 4 illustrates insulating the elements.

FIG. 5 shows a construction with multiple elements.

FIG. 6a demonstrates the use of interlaminar and staggered intralaminar insulators in the same structure. FIG. 6b demonstrates the use of interlaminar and off-set intralaminar insulators in the same structure.

FIG. 7 shows a cross sectional construction of a patient table or device.

FIG. 8 illustrates a modular insert of the present disclosure.

FIG. 9 is an example of a couch top construction using the present disclosure.

FIG. 10 is an example of a modular couch top.

FIG. 11 is an example of a patient positioning device using the present disclosure.

FIG. 12 is an example of a support beam.

### DETAILED DESCRIPTION

The present disclosure described herein can mitigate and/or eliminate the problems of image distortion and localized heating inherent to devices constructed of carbon fiber when used in MR and other RF applications. This will allow the beneficial properties of carbon fiber to be incorporated into devices that can be used in simulation through radiation treatment regardless of the modalities employed (including MR imaging). Fiberglass is typically used in MRI application because it is non-conductive. Fiberglass has a degree of x-ray transparency, however, its attenuation is much greater than carbon fiber. Therefore, it results in poor x-ray signal to noise ratio when laminated into practical thicknesses for patient positioning devices.

Electrical eddy currents occur in conductive material when exposed to a magnetic field because the electrons in the material are able to circulate forming a closed electrical loop. As with electrical wire, the current is conducted down the carbon fiber's length. By embedding a unidirectional set of the conductive carbon fibers in an electrically insulating matrix resin, we can start to take advantage of the anisotropic nature of the composite material's conductivity. That is to say that the conductivity in the fiber direction is orders of magnitude greater than the conductivity transverse to the fibers. This starts to hinder the electrical current's ability to travel up one fiber, cross over, and return down another fiber. A typical electrical conductivity for carbon fiber is about 10⁵ (S/m) whereas the electrical conductivity for epoxy is around 10⁻¹² (S/m). In one embodiment, the conductivity of the conductive ply, layer or lamina is greater than about 10⁴ S/m in the direction of the fibers.

Typical commercially available carbon fiber prepreg materials tend to come in sheets with an areal weight running from about 50 GSM (grams per square meter) up to 1000 GSM. This translates to thicknesses in the range of slightly less than 0.005″ up to 0.025″ or slightly higher. These sheets (also called plies) are layered into a laminate to form structures.

In order to produce RF compatible carbon fiber elements, we need to minimize the ability of the electrons to form eddy currents when placed into the magnetic field. In one embodiment, this can be achieved (1) by producing long carbon fiber composite elements that are very narrow in the transverse direction and (2) by producing short carbon fiber elements that are wide in the transverse direction. The elements are generally composed of conductive fibers oriented in one direction (unidirectional) embedded in an electrically insulating matrix resin. Fabrics comprised of electrically conductive fibers are generally not suitable for these elements as the fabric will create loops in which eddy currents can form. However, a fabric containing a conductive fiber in one direction and a non-conducting fiber in the other direction would be suitable.

These radiofrequency compatible elements can be used as building blocks to produce radiofrequency compatible structures from carbon fiber. However, we must adequately separate and insulate the individual elements from each other so that we do not develop electrical looping paths from one element to the next.

Insulating separators can be included in the structure in several ways. They can be placed in the same plane as the element, (1) separating elements lateral, in the same ply layer, (2) separating elements longitudinally, also in the same ply layer, or (3) in between plies to separate elements through the thickness of the structure. These strategies can be mixed in the same structure to optimize both structural and RF performance.

Insulating elements can be composed of an insulator such as a pure polymer, a polymer with a scrim material (such as non-woven polyester) or a non-conductive composite structural element such as aramid (Kevlar®) so that it contributes to the structural performance as well.

By combining insulating elements with RF compatible carbon fiber elements, a laminate may be produced that is of high structural performance (stiffness and/or strength).

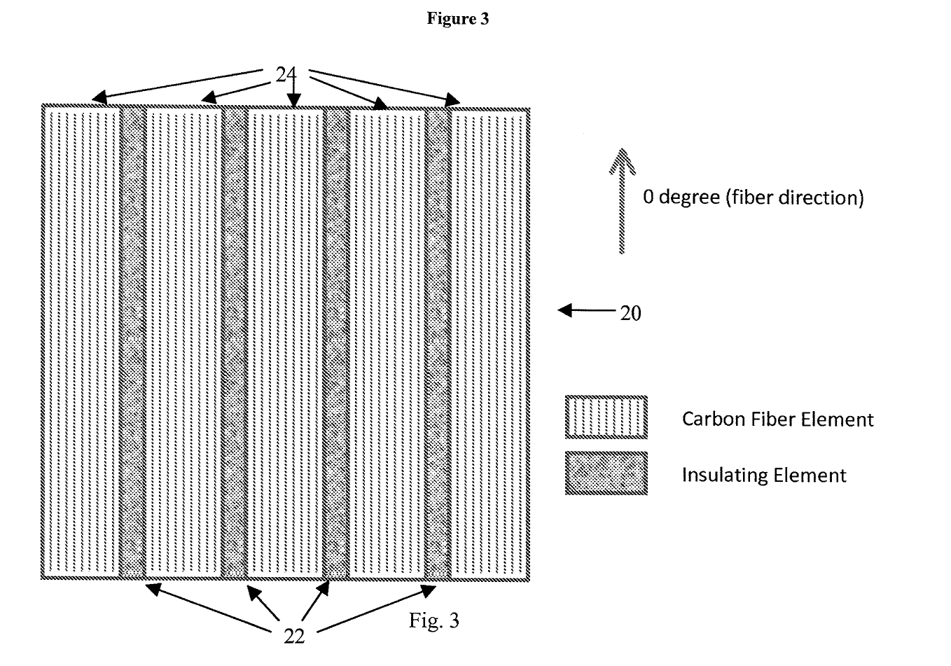

FIG. 3 through FIG. 6 show ways in which conducting elements and insulators can be combined to develop RF compatible lamina (plies). The lamina can then be stacked into a structural laminate that is RF compatible and of high structural performance (stiffness and/or strength). Each lamina can have it's own orientation with respect to the laminate's coordinate system in order to optimize structural performance for any given application.

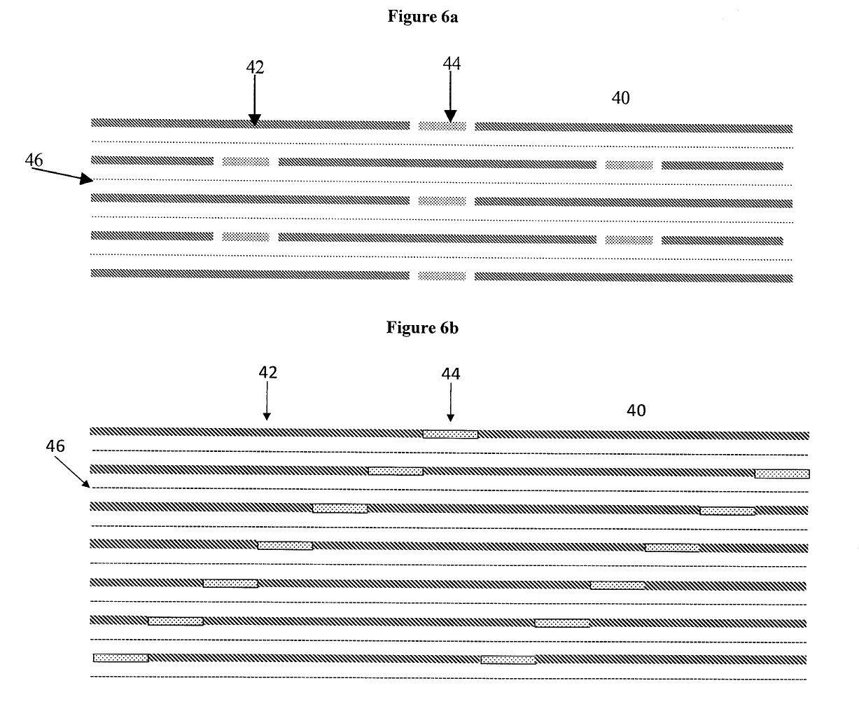

These laminates can be used in any manner typically employed in composite structure design. They can be used to develop solid structures or can be incorporated in typical composite constructions such as sandwich panels. In FIG. 6, a sandwich panel is shown consisting of RF compatible laminate faces placed on a foam core. The edges are wrapped with an insulating composite material so that the top and bottom skin are electrically isolated from each other.

Specifically, the present disclosure provides devices for use in the treatment and simulation of treatment of cancerous tissue that can be used inside a magnetic field used for MR imaging without exhibiting image distortion or local heating. The homogeneity of the structure in an X-ray based environment is also an object of this disclosure so that x-ray artifacting is minimized.

It is another object of the present disclosure to provide devices that can be used with RF technology such as that developed by Calypso without causing interference with the system that would impact treatment.

It is another object of the present disclosure to minimize the signal to noise ratio. The ability to detect an aberrant object in a radiograph is related to the ratio of the differential intensity to the ambient noise level. This ratio is called the absolute contrast to noise ratio, or the image signal to noise ratio. In other words the higher the signal to noise ratio the higher the quality of the image. This has advantages for both diagnosis and treatment simulation as anatomy is more clearly delineated. Noise causes local variations in contrast that does not represent actual attenuation differences in the patient.

The x-ray attenuation of materials is heavily influenced by its atomic structure and element make up. Generally, the higher the atomic mass of the element, the higher the attenuation. Fiberglass is largely composed of Silicon with an atomic mass of ˜28 and Oxygen, with an atomic number of ˜16. Carbon fiber is composed almost entirely of Carbon, which has an atomic mass of ˜12. Aramid fibers are comprised of Carbon, Hydrogen, Oxygen, and Nitrogen. With a lower density than carbon fiber, aramid materials generally have lower x-ray attenuation. Although they lack the structural performance of carbon fiber, they are non-conductive. In a case of varying density across the structure the signal to noise ratio will also vary. This variable signal-to-noise will be seen on the image and will interfere with the operator's ability to diagnose the patient.

In some embodiments, the present disclosure provides a device that is compatible with radiofrequency applications such as magnetic resonance imaging and is also x-ray translucent as shown in the figures. The device is to be constructed of both conductive and non-conductive elements. The conductive elements provide the bulk of the stiffness of the structure. The non-conducting elements are arranged in such a manner to maximize structural performance while at the same time limiting eddy currents in the device. The limiting of the eddy currents is what allows the device to be used in radiofrequency applications.

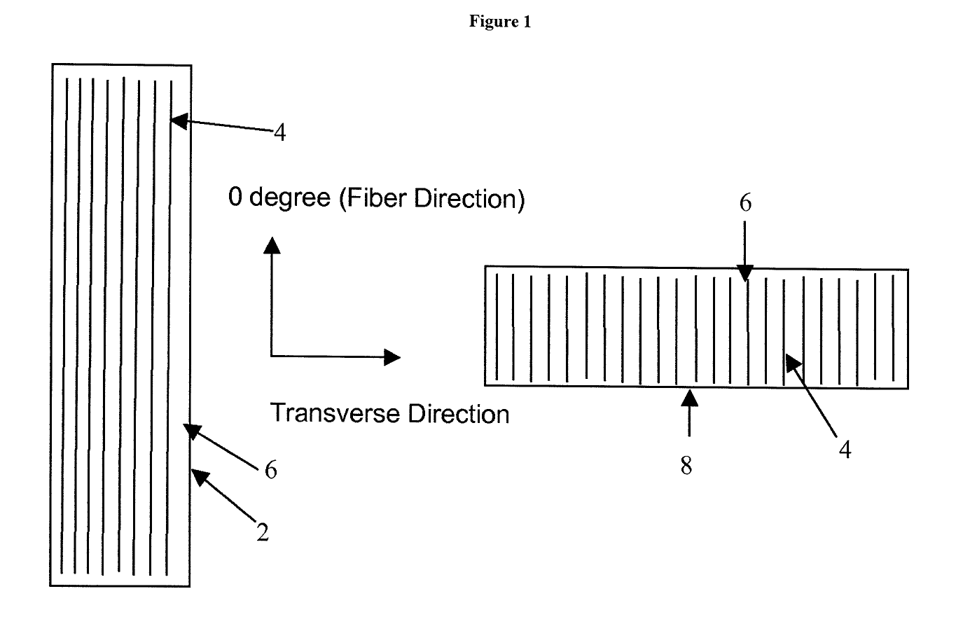

FIG. 1 depicts elements composed of conductive fibers 4 in the 0 degree direction. Conductivity is greatly reduced in the transverse direction as the fibers are embedded in a non-conductive matrix material 6. The element on the left 2 has an aspect ratio that is long in the fiber direction and narrow in the transverse direction. The element on the right 8 is short in the fiber direction and long in the transverse direction. Various aspect ratios may be used to optimize structural performance and minimize electrical conductivity of the system.

In some embodiments, the fibers in each conductive ply, layer or lamina are oriented in substantially the same direction. For example, each fiber can be oriented in the same direction +60 degrees, +45 degrees, +30 degrees, +15 degrees, 0 degrees, −15 degrees, −30 degrees, −45 degrees, −60 degrees. Preferably, the carbon fibers are uniformly distributed in the conductive ply, layer or lamina.

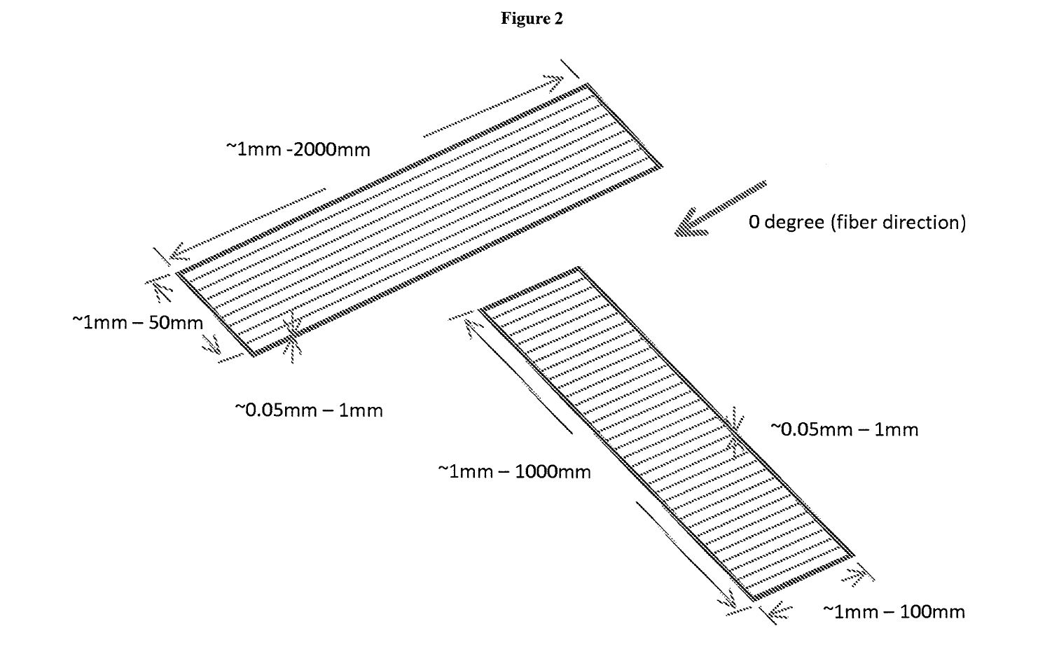

FIG. 2 depicts typical geometries of the elements shown in FIG. 1.

FIG. 3 shows a method of insulating the elements in a lamina 20 by placing multiple conducting elements 24 in a plane (or sheet), separated laterally by insulators 22. By employing a non-conductive element of similar density to the conductive fiber loaded element, homogeneous x-ray performance can be achieved.

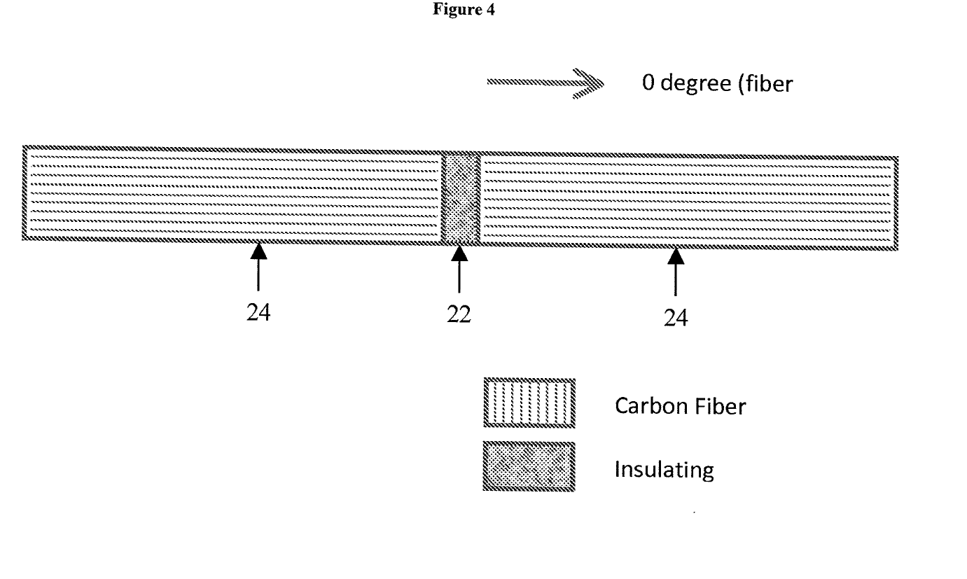

FIG. 4 shows a method of insulating the elements by placing multiple conducting elements 24 in a plane (or sheet), separated longitudinally by insulators 22. By employing a non-conductive element of similar density to the conductive fiber loaded element, homogeneous x-ray performance can be achieved.

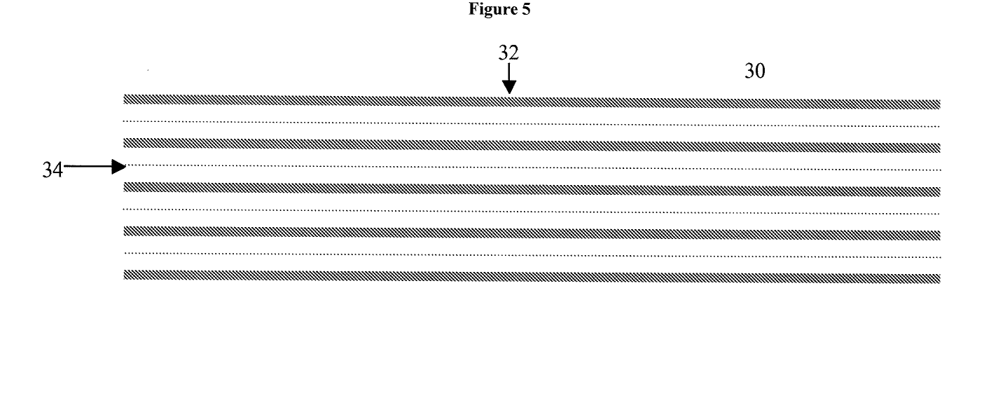

FIG. 5 demonstrates a construction in which sheets of elements (also referred to as plies or lamina) can be layered into a laminate that is compatible with Radio Frequency environments and also x-ray translucent. The 0 degree orientation of each lamina 30 can be placed in any direction with respect to the laminate. In this way, the fiber orientation and structure can be optimized based on the application. An interlaminar insulator 34 is used to separate plies of conducting materials 32 from coming in contact.

FIG. 6a and FIG. 6b demonstrate the use of both interlaminar 46 and intralaminar 44 insulators in the same laminate 40. In FIG. 6a, the joints between conductive 42 and non-conductive elements 44 are staggered to optimize structural performance. In this configuration, the structure is also RF compatible. In particular, structures having a staggered configuration do not present homogeneous attenuation throughout the cross section to beams that are substantially perpendicular to the plane of the laminate. As the x-ray beam is swept across the plane of the laminate it is exposed to a cross section of changing x-ray absorption. Therefore these structures are not homogeneously x-ray compatible. Homogeneously x-ray transparent refers to structures whose attenuation is substantially unchanged at any point along its surface. In FIG. 6b, the joints between the conductive 42 and non-conductive elements 44 are off-set to provide a homogeneously x-ray compatible structure. Structures having an off-set configuration have a substantially uniform or consistent amount of insulating elements in the vertical axis, or along the axis of interrogation (e.g., x-radiation).

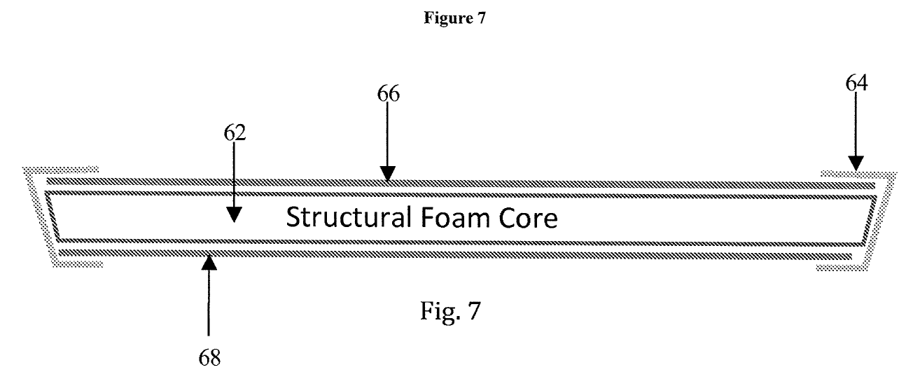

FIG. 7 shows a typical cross sectional construction of a patient table or device that has high structural performance that is RF compatible and x-ray translucent. The top 66 and bottom 68 skins are comprised of lamina as shown in FIG. 6. The top and bottom skins are separated by a non-conductive core 62. In order to maximize the structural integrity, non-conductive materials 64 are wrapped around the edges providing a structural connection between the top and bottom skin. This provides a structural connection without creating an electrical connection.

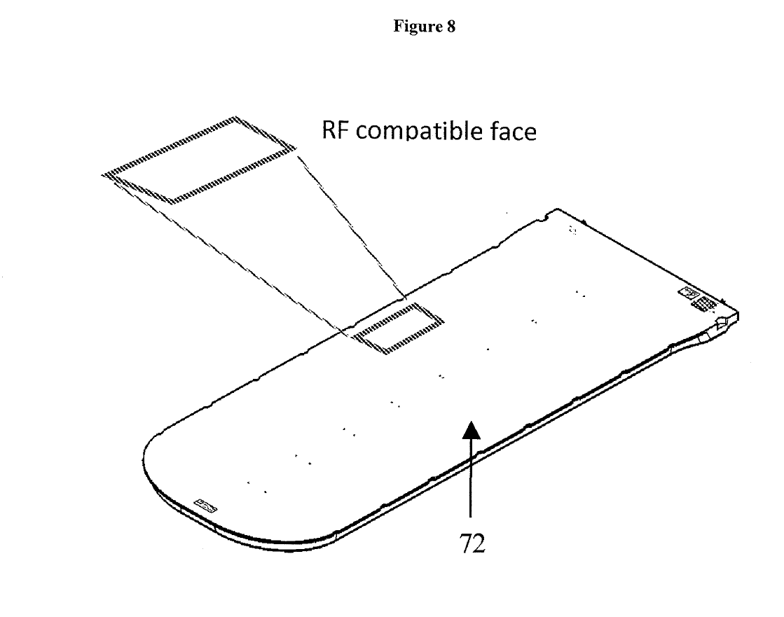

FIG. 8 shows an example of a modular insert 72 for use in radiation therapy constructed in a manner shown in FIG. 7. The modular insert is designed to be used in any imaging or treatment modality.

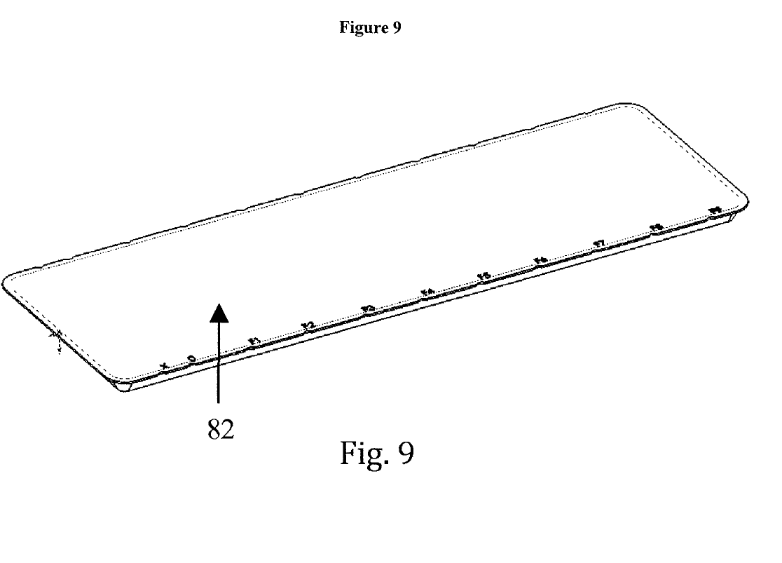

FIG. 9 shows an example of a Monocoque Radiation Therapy Couch Top 82 constructed in the manner shown in FIG. 7. This couch top can be configured for use in any imaging or treatment modality.

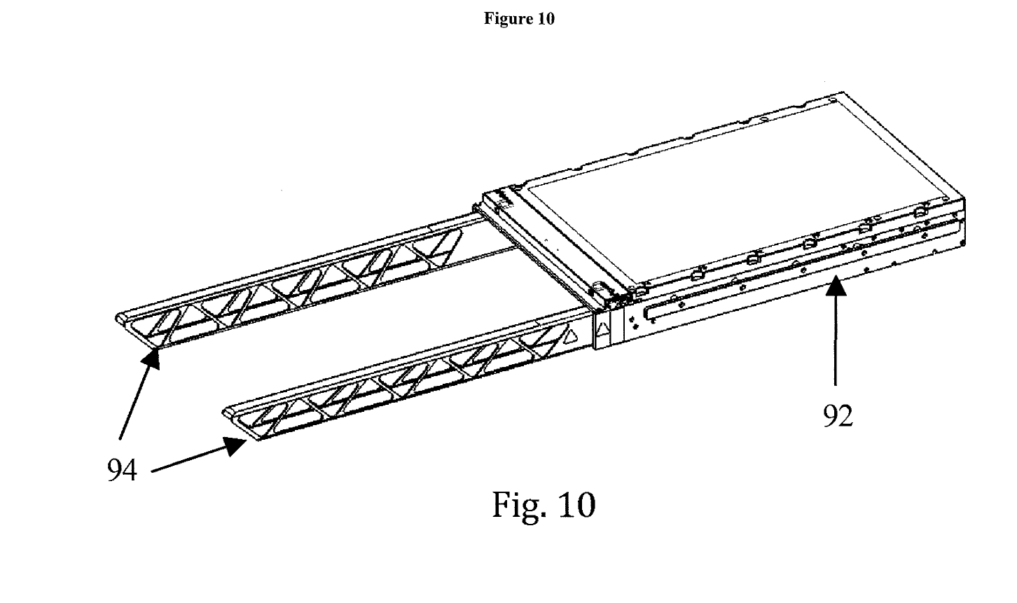

FIG. 10 shows a Modular Radiation Therapy Couch Top 92 that can be used in conjunction with the Modular Insert shown in FIG. 8. The structural support beams 94 are constructed in a manner shown in FIG. 3, FIG. 4, FIG. 5 or FIG. 6.

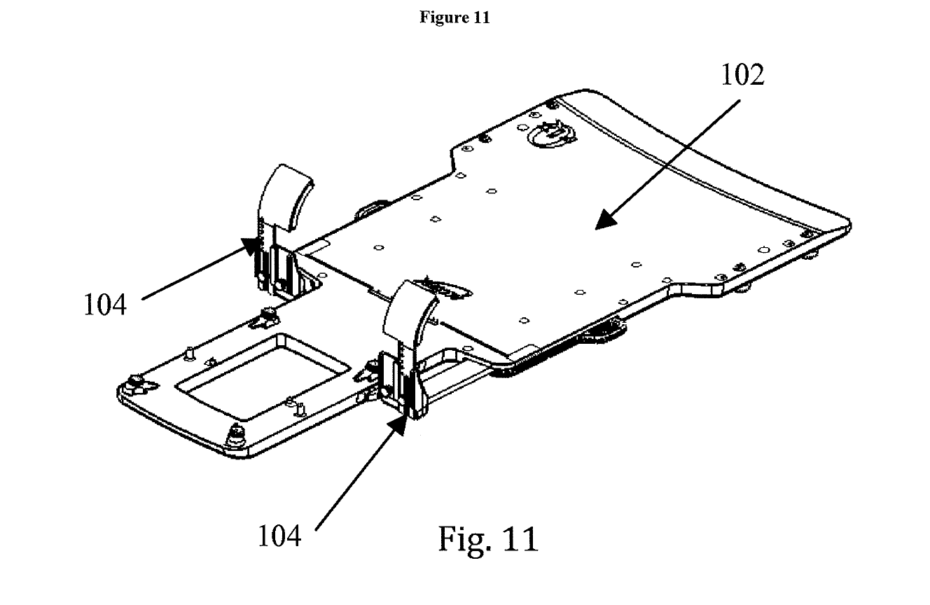

FIG. 11 shows a Patient Positioning Head and Neck Device 102 constructed in the manner shown in FIG. 7. The subcomponents 104 are constructed in any of the manners shown in FIG. 3, FIG. 4, FIG. 5, FIG. 6, and FIG. 7.

The disclosures of all cited references including publications, patents, and patent applications are expressly incorporated herein by reference in their entirety.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention is further defined in the following Examples. It should be understood that these Examples, while indicating preferred embodiments of the invention, are given by way of illustration only.

### EXAMPLES

### Example 1

### Couch Top

The construction of a couch top for use in a radio frequency environment (either in MR or with radio frequency tracking devices) is described. The couch top is constructed of a composite sandwich structure. The core material is an open-cell foam. Other core materials can be used, such as closed-cell foam, honeycomb, wood, or combinations thereof. The couch top has at least a top skin and a bottom skin, preferably both. The top skin and the bottom skin lie on opposite sides of the core and are connected by a non-conductive material. In some embodiments, the connection by a non-conductive material is used to ensure that eddy currents are not created.

The top skin and bottom skin can each have multiple plies of composite material. Building out from the core surface, each skin can have a ply of carbon fiber epoxy with the fibers aligned with the longitudinal axis of the couch top. Next, each skin can have a ply of aramid epoxy composite (or other insulating material) oriented along the longitudinal axis of the couch (i.e., an interlaminar layer, see FIG. 5). This layer provides an insulating layer as well as adding to the stiffness of the couch top. Either layer can be applied first to the core surface. The core surface can be bare or pre-treated or layered with other materials. Another ply of carbon fiber epoxy can be applied next. The fibers may be aligned along or aligned perpendicular to the longitudinal axis of the couch top. Aligning perpendicular adds stiffness in the perpendicular direction. Additional alternating layers of aramid and carbon fiber can be applied in a variety of directions to provide additional directional stiffness. Finally, a layer of non-conductive aramid epoxy woven fabric can be wrapped around the entire couch top. This layer connects the top and bottom skins, provides additional damage tolerance, and can also provide a pleasing aesthetic appearance. In some embodiments, one or more of the non-conductive plies may be slightly longer and/or wider than the conductive layers. The larger sized non-conductive plies can prevent the conductive layers from interacting. The prevention or reduction of such interaction reduces contact between the layers and the creation of eddy current loops. In the couch top, the non-conductive layers are both longer and wider than the conductive layers to ensure that the non-conductive layers completely cover the conductive layers.

The thickness of each ply can vary depending on the strength, stiffness and the insulation required. In some embodiments, each ply can be between about 0.001 inches and about 0.200 inches thick. In other embodiments, each ply can be between about 0.002 inches and about 0.100 inches, or about 0.003 inches and about 0.080 inches, or about 0.004 inches and about 0.060 inches, or about 0.005 inches and about 0.050 inches thick, or about 0.010 inches and about 0.030 inches thick, or any combination of thickness disclosed. In some embodiments, the thickness of the conductive plies can be between about 0.004 inches and about 0.200 inches, and the thickness of the insulating plies can be between about 0.004 inches and about 0.040 inches.

### Example 2

### Couch Top with a Plurality of Plies Per Layer

Similar to example 1, the construction of a couch top for use in a radio frequency environment is described. The couch top has a composite sandwich structure, an open-cell foam core material and a top and a bottom skin. At least one of the top or bottom skins, or both, consist of at least two layers of alternating carbon fiber epoxy and aramid epoxy composite. One or more of the carbon fiber epoxy layers has a plurality of plies of carbon fiber epoxy (e.g., two or more) with all of the carbon fibers of each plurality of plies oriented in substantially the same direction. For example, in one conductive layer having a plurality of plies, the carbon fiber are all oriented substantially perpendicular to the long axis of the couch top. The non-conductive layers or material can fully encompass the conductive layers (e.g., the plurality of conductive plies) to provide insulation and prevent interactions. In embodiments containing at least two layers of conductive plies, one or more of the additional plurality of plies can be oriented in different directions. For example, the second conductive layer having a plurality of plies can have all of the carbon fibers oriented substantially parallel to the long axis of the couch top. Finally, a layer of non-conductive aramid epoxy woven fabric can be wrapped around the entire couch top.

In this example, the multiple plies of conductive material in each layer are permitted to contact, or touch, each other. The plies in contact have their fibers oriented in substantially the same direction. Because the conductivity in the fiber direction is orders of magnitude higher than the conductivity in the transverse direction electrical loops are minimized or not created. Non-conductive layers are positioned to separate plies of conductive material whose fibers are substantially not parallel to each other.

### Example 3

### Couch Top with Intralaminar Elements

Similar to examples 1 and 2, the construction of a couch top for use in a radio frequency environment is described. The couch top has a composite sandwich structure, an open-cell foam core material and a top and a bottom skin. Within each ply of conductive material, additional insulation is provided (i.e., an intralaminar element, see FIG. 6). The additional intralaminar elements provide further reduction of eddy currents and increase radio frequency compatibility.

The width of each intralaminar element may vary depending on the materials used, the thickness and the insulation required. In some embodiments, each element can be between about 0.05 inches and about 12 inches wide. In other embodiments, each element can be' between about 0.07 inches and about 8 inches, or about 0.09 inches and about 6 inches, or about 0.1 inches and about 5.5 inches, or about 0.125 inches and about 5 inches thick, or about 0.5 inches and about 2 inches, or any combination of widths disclosed.

In some embodiments, each skin is constructed with joints between the conductive and non-conductive elements staggered to provide structural performance (See FIG. 6a). In other embodiments, each skin is constructed with joints between the conductive and non-conductive elements off-set to provide both structural performance and homogeneous x-ray translucency (See FIG. 6b). In the off-set arrangement, each skin is constructed such that a cross-section taken at any point in the couch top will show the same amount of conductive and non-conductive material.

### Example 4

### Support Beam

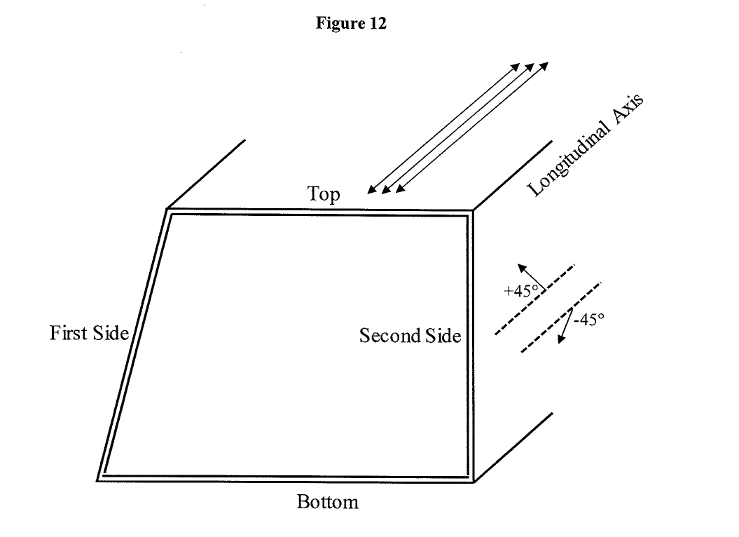

The construction of a support beam for a modular couch top for use in radiation therapy is described. The support beam has a top (e.g., top flange), a bottom (e.g., bottom flange), a first side, and a second side (See FIG. 12). FIG. 12 shows another embodiment of a support beam. The top and bottom of the support beam are designed to provide bending stiffness along the longitudinal axis of the support beam. The top and bottom can contain a plurality of plies of carbon fiber epoxy composites, either in individual layers or grouped in different layers. One or more of the plies contain fibers oriented along the longitudinal axis of the beam to provide the longitudinal stiffness. In some embodiments, a majority of the plies have fibers oriented along the longitudinal axis. In other embodiments, all of the plies have fibers oriented along the longitudinal axis. Dispersed amongst these plies are non-conductive plies. The arrangement of the conductive and non-conductive plies can be any arrangement disclosed in either examples 1-3 (i.e., interlaminar and/or intralaminar elements, a plurality of plies, etc.). In one embodiment, the non-conductive plies are longer and/or wider than the conductive plies, and wrap over and encompass the conductive plies.

The first and second sides are designed to provide torsional stiffness and to serve as webs connecting the top and bottom. The first and second sides also contain a plurality of plies of carbon fiber epoxy composites, and non-conductive plies as needed. The arrangement of the conductive and non-conductive plies can be any arrangement disclosed in either examples 1-3 (i.e., interlaminar and/or intralaminar elements, a plurality of plies, etc.). In one embodiment, to provide additional torsional stiffness, each side contains at least two carbon fiber epoxy composites plies (or layers) separated by a non-conductive ply wherein the carbon fibers of the conductive plies are oriented +45° and −45°, respectively, to the long axis of the beam (See FIG. 12).

In some embodiments, one or more of the non-conductive plies may be slightly longer and/or wider than the conductive layers. The larger sized non-conductive plies can prevent the conductive layers from interacting. The prevention or reduction in such interaction reduces contact between the layers and the creation of eddy current loops.

To connect the top, bottom, first side, and second side together the support beam may be wrapped with non-conductive plies. These plies may be in the form of woven fabrics or unidirectional material.

While this disclosure has been particularly shown and described with reference to example embodiments thereof, it will be understood by those skilled in the art that various changes in form and details may be made therein without departing from the scope of the invention encompassed by the appended claims.

We claim:

1. A structure comprising
(i) at least two electrically conductive lamina having carbon fibers embedded in a non-conductive matrix, wherein each conductive lamina has an axis perpendicular to the plane of the lamina, and
(ii) at least one insulating lamina having an axis perpendicular to the plane of the lamina, wherein the conductive lamina are separated by the insulating lamina along the axis perpendicular to the plane of the lamina, wherein the structure is x-ray translucent and does not significantly affect magnetic resonance imaging, x-ray based imaging or other radiofrequency dependent applications.

2. The structure of claim 1 wherein the non-conductive matrix comprises epoxy, polyester, vinylester, or ceramic.

3. The structure of claim 1 wherein the insulating lamina comprises aramid, ultra-high-molecular-weight polyethylene or fiberglass.

4. The structure of claim 1, wherein the x-ray based imaging comprises RF Localization, radiation therapy treatment or diagnostic imaging.

5. A structure comprising
(i) at least two electrically conductive lamina having carbon fiber elements embedded in a non-conductive matrix and insulating elements, wherein each conductive lamina has an axis perpendicular to the plane of the lamina and a zero degree in plane axis and a ninety degree in plane axis, wherein the carbon fiber elements are separated by the insulating elements along at least one of the zero degree axis and the ninety degree axis, and
(ii) at least one insulating lamina having an axis perpendicular to the plane of the lamina, wherein the conductive lamina are separated by the insulating lamina along the axis perpendicular to the plane of the lamina, wherein the structure is homogeneously x-ray translucent and does not significantly affect magnetic resonance imaging, x-ray based imaging or other radiofrequency dependent applications.

6. The structure of claim 5, wherein the insulating elements in each conductive lamina are off-set from each other in at least one of the zero degree axis and the ninety degree axis such that there are an equal number of insulating elements through the axis perpendicular to the plane of the lamina.

7. The structure of claim 5 wherein the non-conductive matrix comprises epoxy, polyester, vinylester, or ceramic.

8. The structure of claim 5 wherein the insulating lamina comprises aramid, ultra-high-molecular-weight polyethylene or fiberglass.

9. The structure of claim 5, wherein the x-ray based imaging comprises RF Localization, radiation therapy treatment or diagnostic imaging.

10. A patient positioning device comprising a core, a top face and a bottom face, wherein at least the top or bottom face includes a structure of claim 1.

11. A support beam comprising a top, a bottom, a first side, a second side and a longitudinal axis, wherein at least the top or bottom includes a structure of claim 1, and wherein at least one of the sides includes a structure of claim 1.

12. A method of preparing a patient positioning device, the method comprising:
(i) placing on a core at least two electrically conductive lamina having carbon fibers embedded in a non-conductive matrix, wherein each conductive lamina has an axis perpendicular to the plane of the lamina, and
(ii) placing on the core at least one insulating lamina having an axis perpendicular to the plane of the lamina, wherein the conductive lamina are separated by the insulating lamina along the axis perpendicular to the plane of the lamina, wherein the structure is X-ray translucent,
wherein the device does not interfere with magnetic resonance and radiofrequency based diagnostics.

13. The method of claim 13, wherein the device reduces or eliminates image distortion, local heating or combinations thereof.
